(19) United States
(12) Patent Application Publication
(10) Pub. No.: US 2012/0009137 A1
(43) Pub. Date: Jan. 12, 2012

(54) ANTIPERSPIRANT COMPOSITIONS WITH WHITER COLOR AND ENHANCED SKIN FEEL AND METHODS FOR MANUFACTURING THE SAME

(75) Inventor: Travis T. Yarlagadda, Phoenix, AZ (US)

(73) Assignee: The Dial Corporation, Scottsdale, AZ (US)

(21) Appl. No.: 12/833,153

(22) Filed: Jul. 9, 2010

(51) Int. Cl. A61K 8/58; A61Q 15/00

(52) U.S. Cl. 424/66

(57) ABSTRACT

Antiperspirant products with whiter color and enhanced skin feel and methods for manufacturing the same are provided. In an embodiment, an antiperspirant product comprises an active antiperspirant compound and linoleamidopropyl PG-dimonium chloride phosphate dimethicone. The antiperspirant product has an L* value according to the CIE L*a*b* color scale of at least 93.

### FIELD OF THE INVENTION

The present invention generally relates to antiperspirant products and methods for manufacturing antiperspirant products, and more particularly relates to antiperspirant products with whiter color and enhanced skin feel and methods for manufacturing the same.

### BACKGROUND OF THE INVENTION

Antiperspirants are popular personal care products used to prevent or eliminate perspiration and body odor caused by perspiration. Antiperspirant sticks are desired by a large majority of the population because of the presence of active antiperspirant compounds that minimize or prevent the secretion of perspiration by blocking or plugging ducts of sweat-secreting glands, such as those located at the underarms. Antiperspirants typically comprise an active antiperspirant compound in a carrier that permits the antiperspirant product to be applied to the skin by swiping or rubbing the stick across the skin, typically of the underarm. Upon application, the carrier evaporates, releasing the active antiperspirant compound from the antiperspirant product to form plugs in the sweat ducts.

However, antiperspirant users often are disappointed in the visual aesthetics and the skin feel of present-day antiperspirants. Many conventional antiperspirant products exhibit a yellow cast that is typically due, at least in part, to the active antiperspirant compounds in the antiperspirant products. Antiperspirant users generally prefer whiter antiperspirant products because the color white tends to convey images of cleanliness and health. In addition, antiperspirant users often find antiperspirant products abrasive and/or irritating to the skin and are disappointed in the chalky, brittle, and/or crumbly application of the solid wax stick across the skin.

Accordingly, it is desirable to provide antiperspirant products that are whiter in color than conventional antiperspirant sticks. In addition, it is desirable to provide antiperspirant products with enhanced skin feel. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant products with whiter color and enhanced skin feel and methods for manufacturing the same are provided. In accordance with an exemplary embodiment, an antiperspirant product comprises an active antiperspirant compound and linoleamidopropyl PG-dimonium chloride phosphate dimethicone. The antiperspirant product has an L* value according to the CIE L*a*b* color scale of at least 93.

In accordance with another exemplary embodiment, a method of manufacturing an antiperspirant product comprises combining an active antiperspirant compound and linoleamidopropyl PG-dimonium chloride phosphate dimethicone at a first temperature to form a mixture. The mixture is poured into molds at a second temperature that is lower than the first temperature. The mixture is cooled to a third temperature that is lower than the second temperature. The cooled mixture has an L* value according to the CIE L*a*b* color space of at least 93.

### BRIEF DESCRIPTION OF THE DRAWINGS

The present invention will hereinafter be described in conjunction with the following drawing figures, wherein like numerals denote like elements, and wherein:

FIG. 1 is a schematic illustration of the CIE L*a*b* color space.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to an antiperspirant product that is whiter in color than conventional antiperspirant products. Various embodiments also exhibit better “skin feel”, that is, when applied to the skin of a user, the antiperspirant product exhibits reduced caking and crumbly residue, reduced slipperiness, that is, reduced slip between the underarms, and improved glide. The term “glide” typically is used to denote the perceived friction between the antiperspirant product and the skin. The smoother the glide, or the less friction between the product and the skin, the more desirable the product is to users. It unexpectedly has been found that antiperspirant products that are whiter in color and/or exhibit improved skin feel can be achieved when manufactured to contain linoleamidopropyl PG-dimonium chloride phosphate dimethicone (LPCPD), a synthetic phospholipid. One example of such a phospholipid is that available under the trade name Arlasilk™ Phospholipid PLN available from Croda Inc. of Edison, N.J. In an exemplary embodiment, the antiperspirant product comprises LPCPD in an amount of up to 4.5 weight percent (wt. %).

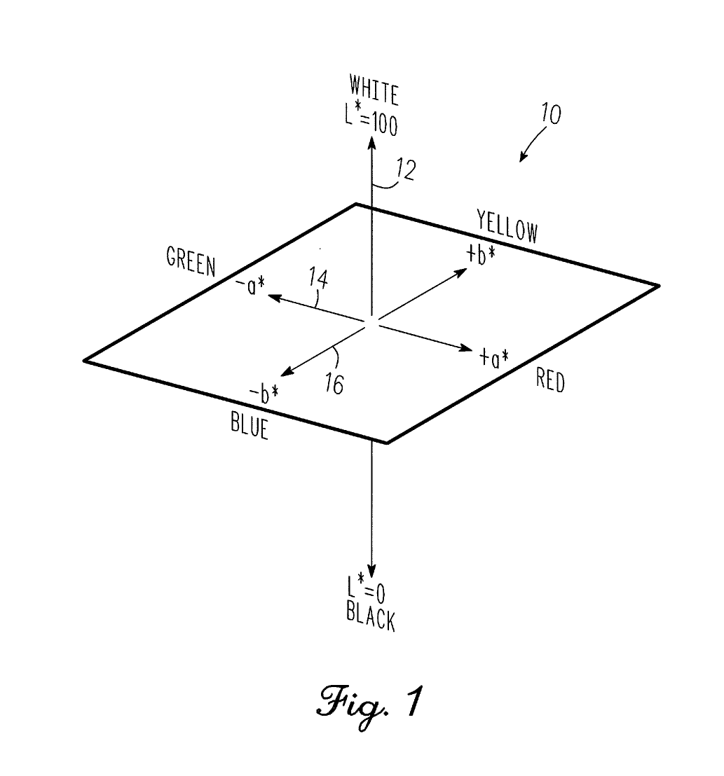

As noted above, it has been unexpectedly found that antiperspirant products, such as those with the formulas contemplated herein, that contain LPCPD are whiter in color than conventional antiperspirant colors. The whiteness of an object can be measured according to the well-known CIE L*a*b* color scale 10, illustrated in FIG. 1. The CIE L*a*b* color scale is an approximately uniform color scale. In a uniform color scale the differences between points in the color space correspond to visual differences between the colors plotted. The CIE L*a*b* color space is organized in a cube form. The L* axis 12 runs from top to bottom. The maximum L* is 100, which represents a perfect reflecting diffuser, i.e., the color white. The minimum for L* is zero, which represents black. The a* and b* axis, 14 and 16 respectively, have no specific numerical limits. Positive a* is red. Negative a* is green. Positive b* is yellow. Negative b* is blue.

The antiperspirant products contemplated herein containing LPCPD exhibit a whiter color, that is, a greater L* value, than comparable antiperspirant products that do not contain LPCPD. As used herein, the term “comparable antiperspirant products” means conventional antiperspirant products that otherwise contain the same ingredients as the antiperspirant products contemplated herein but that do not contain LPCPD. The antiperspirant products contemplated herein may also have an a* value and/or a b* value that is closer to zero than comparable antiperspirant products. In one exemplary embodiment, the antiperspirant products contemplated herein have an L* value of at least 93. In a preferred embodiment, the antiperspirant products contemplated herein have an L* value of at least 94. In another embodiment, the antiperspirant products contemplated herein have an a* value of no greater than the absolute value of 4.5. In yet another embodiment, the antiperspirant products contemplated herein have a b* value of no greater than the absolute value of 13.

The various embodiments of the antiperspirant products also comprise a water-soluble active antiperspirant compound. Active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used in the antiperspirant product include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; a and b total to about 6; x is from 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zirconyl hydroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the active antiperspirant compound is aluminum zirconium pentachlorohydrex glycine complex or aluminum zirconium trichlorohydrex glycine complex. In a more preferred embodiment, the antiperspirant product comprises an active antiperspirant compound in an amount of about 8 to about 30 wt. % (USP). As used herein, weight percent (USP) or wt. % (USP) of an antiperspirant salt is calculated as anhydrous weight percent in accordance with the U.S.P. method, as is well known in the art. This calculation excludes any bound water and glycine. In a most preferred embodiment, the antiperspirant product comprises about 20-25 wt. % aluminum zirconium pentachlorohydrex glycine complex or aluminum zirconium trichlorohydrex glycine complex.

Further included in the antiperspirant product is at least one structurant and/or gellant (hereinafter referred collectively as “structurant”) that facilitates the solid consistency of the antiperspirant stick product. Naturally-occurring or synthetic waxy materials or combinations thereof can be used as such structurants. Suitable structurants, including waxes and gellants, are often selected from fatty alcohols often containing from 12 to 30 carbons, such as stearyl alcohol, behenyl alcohol and sterols such as lanosterol. As used herein, the term “fatty” means a long chain aliphatic group, such as at least 8 or 12 linear carbons, which is frequently not branched (linear) and is typically saturated, but which can alternatively be branched and/or unsaturated. It is possible for the fatty acid to contain a hydroxyl group, as in 12-hydroxystearic acid, for example as part of a gellant combination, and to employ amido or ester derivates thereof.

Other structurants can comprise hydrocarbon waxes such as paraffin waxes, microcrystalline waxes, ceresin, squalene, and polyethylene waxes. Other suitable structurants are waxes derived or obtained from plants or animals such as hydrogenated castor oil, hydrogenated soybean oil, carnabau, spermacetti, candelilla, beeswax, modified beeswaxes, and Montan wax and individual waxy components thereof. It is especially suitable herein to employ a mixture of wax structurants. Suitable mixtures of structurants can reduce the visibility of active antiperspirant compounds deposited on the skin and result in either a soft solid or a firm solid. In an exemplary embodiment, the surfactant(s) comprise about 10 to about 35 wt. % of the total antiperspirant product. In a preferred embodiment, the antiperspirant product comprises a mixture of stearyl alcohol and hydrogenated castor oil. In a more preferred embodiment, the antiperspirant product comprises about 12 to about 25 wt. % stearyl alcohol and about 1.5 to about 7 wt. % hydrogenated castor oil. In a most preferred embodiment, the antiperspirant product comprises about 15-22 wt. % stearyl alcohol and about 2.8 wt. % hydrogenated castor oil.

The antiperspirant products also may comprise a high refractive index (R.I.) hydrophobic compound. As used herein, the term “high refractive index” means a refractive index of no less than about 1.4. The high R.I. hydrophobic compound also facilitates the minimization and/or prevention of a white residue on the skin by masking the active antiperspirant salt that stays upon the skin upon evaporation of the carrier. Examples of high R.I. hydrophobic compounds for use in the antiperspirant products include PPG-14 butyl ether, C₁₂-C₁₅ alkyl benzoate, such as Finsolv TN® available from Innospec of the United Kingdom, and phenyl dimethicone. In a preferred embodiment, the antiperspirant product comprises PPG-14 butyl ether and, in a more preferred embodiment, the antiperspirant product comprises PPG-14 butyl ether in an amount of about 5 to about 15 wt. % of the total antiperspirant product. In a most preferred embodiment, the antiperspirant product comprises about 9 to about 11 wt. % PPG-14 butyl ether.

In another exemplary embodiment, the antiperspirant product comprises one or more suspending agents that facilitate suspension of the active antiperspirant compound in the antiperspirant product, thereby minimizing the amount of active antiperspirant compound that settles out of the antiperspirant product during manufacture. Suitable suspending agents include clays and silicas. Examples of suitable silicas include fumed silicas and silica derivatives, such as silica dimethyl silylate. Suitable clays include bentonites, hectorites and colloidal magnesium aluminum silicates. In one exemplary embodiment, the antiperspirant product comprises about 0.2 to about 2.5 wt. % suspending agents. In another exemplary embodiment, the antiperspirant product comprises a mixture of silica and silica dimethyl silylate. In a preferred embodiment, the antiperspirant product comprises from about 0.1-0.5 wt. % silica and from about 0.1 to about 2 wt. % silica dimethyl silylate. In another exemplary embodiment, the antiperspirant product does not use suspending agents, but comprises high melting point waxes to prevent settling of the active ingredients. Examples of suitable high melting point waxes include hydrogenated castor oils and polyethylenes having various melting points above 65° C.

In addition to the ingredients identified above, the antiperspirant product may comprise additives, such as those used in conventional antiperspirants. These additives include, but are not limited to, fragrances, including encapsulated fragrances, dyes, pigments, preservatives, antioxidants, moisturizers, and the like. These optional ingredients can be included in the antiperspirant product in an amount of 0 to about 20 wt. %. In a preferred embodiment, the antiperspirant product comprises myristyl myristate, which provides a conditioning effect to the skin.

The antiperspirant product further comprises at least one hydrophobic carrier. An example of suitable hydrophobic carriers includes liquid siloxanes and particularly volatile polyorganosiloxanes, that is, liquid materials having a measurable vapor pressure at ambient conditions. The polyorganosiloxanes can be linear or cyclic or mixtures thereof. The linear volatile silicones generally have viscosities of less than about 5 centistokes at 25° C., while the cyclic volatile silicones have viscosities under 10 centistokes. Preferred siloxanes include cyclomethicones, which have from about 3 to about 6 silicon atoms, such as cyclotetramethicone, cyclopentamethicone, and cyclohexamethicone, and mixtures thereof. The carrier also may comprise, additionally or alternatively, nonvolatile silicones such as dimethicone and dimethicone copolyols, which have from about 2 to about 9 silicon atoms. Examples of suitable dimethicone and dimethicone copolyols include polyalkyl siloxanes, polyalkylaryl siloxanes, and polyether siloxane copolymers.

The antiperspirant product, according to various embodiments, can be prepared by combining the active antiperspirant compound and LPCPD at 65-75° C. to form a mixture, pouring the mixture into molds at about 53° C., and cooling the mixture to room temperature. In a preferred embodiment, the antiperspirant product is prepared by combining the suspending agents in the carrier. Any suitable form of mixing can be used to combine the ingredients, such as high shear mixing, stirring, agitation, blending, or any combination thereof. The active antiperspirant compound is added to the suspending agents and carrier to form a premix. Mixing continues until the premix is homogenous and fluid in consistency. The structurants and the high refractive index (R.I.) hydrophobic compound, if used, are added to a mixing vessel and heat not exceeding 85° C. is applied to melt the ingredients. As the ingredients melt, agitation is slowly commenced. Once the mixture is molten, it is cooled to 65-75° C. and LPCPD is added. The mixture, if necessary, is further cooled to 64-69° C. and, with continuous agitation, the premix is incrementally added to the mixture until the mixture is homogenous. Additional carrier is added to the mixture with agitation such that the mixture is maintained at a temperature of 60° C. Additives, such as fragrance, dyes, corn starch, etc. are added with mixing while maintaining the mixture at 60° C. The final mixture is cooled to 53° C., poured into molds, and then allowed to cool to room temperature. As used herein, the teem “allowed to cool” means exposing the mixture to room temperature for a time sufficient for the mixture to come to room temperature or exposing the mixture to a refrigerator or cooling room, fan, or other cooling mechanism that lowers the temperature of the mixture to room temperature. In another embodiment, the phospholipid can be added to the premix with high sheer mixing to form the homogenous premix and the premix can be added to the molten mixture as described above. A portion of the phospholipid can be added to the premix in addition to a portion being added directly to the molten mixture, or the entire amount of the phospholipid can be added to the premix as an alternative to the addition to the molten mixture. It will be appreciated that the sequence of addition and/or combination of the various components of the antiperspirant product is not necessarily critical, and various sequences for addition or combination of the components can be used.

The following are exemplary embodiments of an antiperspirant product contemplated herein, with each of the components set forth in weight percent of the antiperspirant product. The examples are provided for illustration purposes only and are not meant to limit the various embodiments of the antiperspirant product in any way.

### Example 1

| Ingredient | Wt. % |
|---|---|
| Cyclopentasiloxane | 35.88 |
| Aluminum zirconium | 22.20 |
| pentachlorohydrex GLY | |
| Stearyl Alcohol | 20.43 |
| PPG-14 butyl ether | 11.00 |
| Hydrogenated castor oil | 2.84 |
| Zea mays corn starch | 0.27 |
| Arlasilk ™ Phospholipid PLN | 2.00 |
| Myristyl Myristate | 1.92 |
| Silica | 0.16 |
| Silica dimethyl silylate | 0.65 |
| Dye | 0.02 |
| Fragrance | 2.63 |
| Total | 100.00 |

### Example 2

| Ingredient | Wt. % |
|---|---|
| Cyclopentasiloxane | 37.28 |
| Aluminum zirconium | 21.84 |
| pentachlorohydrex GLY | |
| Stearyl Alcohol | 20.00 |
| PPG-14 butyl ether | 9.80 |
| Hydrogenated castor oil | 2.84 |
| Arlasilk ™ Phospholipid PLN | 3.00 |
| Myristyl Myristate | 1.92 |
| Silica | 0.34 |
| Silica dimethyl silylate | 1.38 |
| Fragrance | 1.60 |
| Total | 100.00 |

The antiperspirant product of Example 2 was prepared by adding 712.42 grams (g) of cyclopentasiloxane to a mixing vessel and commencing agitation. With agitation, 7.12 g silica and 28.6 g silica dimethyl silylate were incrementally added to the cyclopentasiloxane and agitation continued until the silica was wetted. Utilizing a high shear mixer, 451.98 g aluminum zirconium pentachlorohydrex glycine complex was added to the suspending agents and carrier to form a premix. Mixing continued until the premix was homogenous and fluid in consistency. 300 g stearyl alcohol, 42.6 g hydrogenated castor oil, 147 g PPG-14 butyl ether, and 28.8 g myristyl myristate were added to a mixing vessel and heat not exceeding 85° C. was applied to melt the ingredients. As the ingredients melted, agitation was slowly commenced. Once the mixture was molten, it was cooled to 65-75° C. and 45 g of LPCPD was added. The mixture, if necessary, was further cooled to 64-69° C. and, with continuous agitation, the premix was incrementally added to the mixture until the mixture was homogenous. 42.6 g cyclopentasiloxane was added to the mixture with agitation such that the mixture was maintained at a temperature of 60° C. 24 g fragrance was added with mixing while maintaining the mixture at 60° C. The final mixture was cooled to 53° C., poured into molds, and then allowed to cool to room temperature.

Accordingly, various embodiments of antiperspirant products containing linoleamidopropyl PG-dimonium chloride phosphate dimethicone have been provided. The antiperspirant products are whiter in color than conventional antiperspirant products without linoleamidopropyl PG-dimonium chloride phosphate dimethicone. Various embodiments also exhibit better “skin feel”, that is, when applied to the skin of a user, the antiperspirant product exhibits reduced caking and crumbly residue, reduced slipperiness, and improved glide.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant product comprising:
an active antiperspirant compound; and
linoleamidopropyl PG-dimonium chloride phosphate dimethicone,
wherein the antiperspirant product has an L* value according to the CIE L*a*b* color scale of at least 93.

2. The antiperspirant product of claim 1, wherein the antiperspirant product has an L* value of at least 94.

3. The antiperspirant product of claim 1, wherein the antiperspirant product has an a* value according to the CIE L*a*b* color scale of no greater than an absolute value of 4.5.

4. The antiperspirant product of claim 1, wherein the antiperspirant product has a b* value according to the CIE L*a*b* color scale of no greater than an absolute value of 13.

5. The antiperspirant product of claim 1, wherein linoleamidopropyl PG-dimonium chloride phosphate dimethicone is present in an amount of no greater than about 4.5 wt. % of the antiperspirant product.

6. The antiperspirant product of claim 1, wherein the active antiperspirant compound is aluminum zirconium trichlorohydrex glycine complex.

7. The antiperspirant product of claim 1, wherein the active antiperspirant compound is aluminum zirconium pentachlorohydrex glycine complex.

8. The antiperspirant product of claim 1, further comprising a hydrophobic carrier.

9. The antiperspirant product of claim 8, wherein the hydrophobic carrier is cyclopentasiloxane.

10. The antiperspirant product of claim 1, further comprising a mixture of stearyl alcohol and hydrogenated castor oil.

11. The antiperspirant product of claim 1, further comprising a high refractive index hydrophobic compound.

12. A method of manufacturing an antiperspirant product, the method comprising the steps of:
combining an active antiperspirant compound and linoleamidopropyl PG-dimonium chloride phosphate dimethicone at a first temperature to form a mixture;
pouring the mixture into molds at a second temperature that is lower than the first temperature; and
cooling the mixture to form a cooled mixture having a third temperature that is lower than the second temperature, wherein the cooled mixture has an L* value according to the CIE L*a*b* color space of at least 93.

13. The method of claim 12, wherein the step of cooling comprises cooling the mixture, wherein the cooled mixture has an L* value of at least 94.

14. The method of claim 12, wherein the step of combining comprises combining the active antiperspirant compound and linoleamidopropyl PG-dimonium chloride phosphate dimethicone in an amount of up to 4.5 wt. % of the antiperspirant product.

15. The method of claim 12, wherein the step of combining comprises combining aluminum zirconium trichlorohydrex glycine complex and linoleamidopropyl PG-dimonium chloride phosphate dimethicone.

16. The method of claim 12, wherein the step of combining comprises combining aluminum zirconium pentachlorohydrex glycine complex and linoleamidopropyl PG-dimonium chloride phosphate dimethicone.

17. The method of claim 12, wherein the step of combining comprises combining the active antiperspirant compound, linoleamidopropyl PG-dimonium chloride phosphate dimethicone, and a carrier.

18. The method of claim 17, wherein the step of combining comprises combining the active antiperspirant compound, linoleamidopropyl PG-dimonium chloride phosphate dimethicone, and cyclopentasiloxane.

19. The method of claim 12, wherein the step of combining comprises combining the active antiperspirant compound, linoleamidopropyl PG-dimonium chloride phosphate dimethicone, stearyl alcohol and hydrogenated castor oil.

20. The method of claim 12, wherein the step of combining comprises combining the active antiperspirant compound, linoleamidopropyl PG-dimonium chloride phosphate dimethicone, and a high refractive index hydrophobic compound.
